(19) United States
(12) Patent Application Publication
(10) Pub. No.: US 2021/0198174 A1
(43) Pub. Date: Jul. 1, 2021

(54) PROCESS FOR MAKING HIGH PURITY SALTS

(71) Applicant: Milliken & Company, Spartanburg, SC (US)

(72) Inventor: Darin L. Dotson, Moore, SC (US)

(21) Appl. No.: 17/119,070

(22) Filed: Dec. 11, 2020

Related U.S. Application Data
(60) Provisional application No. 62/954,714, filed on Dec. 30, 2019.

(51) Int. Cl. C07C 51/50; C07C 51/43

(52) U.S. Cl. CPC C07C 51/50; C07C 61/22; C07C 51/43

(57) ABSTRACT

A process for making a high purity salt comprises the steps of providing an organic compound, providing a metal salt, adding the metal salt and organic compound to an aqueous medium, heating the reaction mixture to react the organic compound and the metal salt to form an organic salt, collecting the organic salt, and directly contacting the collected organic salt with a heated gas stream while agitating the collected organic salt to reduce the moisture content of the dried organic salt to about 7% or less.

### RELATED APPLICATIONS

This application claims priority to co-pending U.S. Provisional Patent Application 62/954,714 filed on Dec. 30, 2019, which is herein incorporated by reference.

### TECHNICAL FIELD OF THE INVENTION

This patent application relates to a process for making high purity salts. In particular, the patent application describes a method for making salts of cis-cyclohexane-1,2-dicarboxylic acid with low levels of trans-cyclohexane-1,2-dicarboxylate salts.

### BACKGROUND

Salts produced in aqueous media typically are dried to a relatively low moisture content prior to further use. The processes used to dry such salts typically are optimized to maximize throughput. In other words, the drying conditions are selected to dry the maximum amount of salt in a minimum amount of time. Such rapid drying of the salt can have unintended consequences. For instance, high heat can lead to degradation of some of the salt, decreasing the overall purity of the final product. Also, excessive heat can cause rearrangement reactions in certain salts, especially in salts of organic acids (organic salts). These rearrangement reactions will affect the isomeric purity (e.g., stereoisomeric purity) of the resulting salt. And changes in the isomeric purity of the salt can render it unsuitable for certain uses, such as pharmaceutical or food contact uses.

A need therefore remains for a process for producing salts (e.g., salts of organic acids) that achieves acceptable throughput while maintaining the isomeric purity (e.g., stereoisomeric purity) of the final product. The process described herein is believed to meet such need.

### BRIEF SUMMARY OF THE INVENTION

In a first embodiment, the invention provides a process for making a high purity salt, the process comprising the steps of:

(a) providing an organic compound selected from the group consisting of cis-cyclohexane-1,2-dicarboxylic acid anhydride, cis-cyclohexane-1,2-dicarboxylic acid, and mixtures thereof;

(b) providing a metal salt selected from the group consisting of metal hydroxides, metal carbonates, metal nitrates, metal chlorides, metal carboxylates, the metal salt comprising a metal selected from the group consisting of alkali metals and alkaline earth metals;

(c) adding the metal salt and the organic compound to an aqueous medium to produce a reaction mixture;

(d) heating the reaction mixture to a temperature of about 65° C. to about 80° C. to react the organic compound and the metal salt and form an organic salt;

(e) collecting the organic salt from the aqueous medium;

(f) directly contacting the collected organic salt with a heated gas stream while agitating the collected organic salt to reduce the moisture content of the dried organic salt to about 7% or less.

In another embodiment, the invention process a process for making a high purity salt, the process comprising the steps of:

(a) providing an organic compound selected from the group consisting of cis-cyclohexane-1,2-dicarboxylic acid anhydride, cis-cyclohexane-1,2-dicarboxylic acid, and mixtures thereof;

(b) providing a calcium salt;

(c) adding the calcium salt and the organic compound to an aqueous medium to produce a reaction mixture;

(d) heating the reaction mixture to a temperature of about 65° C. to about 80° C. to react the organic compound and the metal salt and form an organic salt;

(e) collecting the organic salt from the aqueous medium;

(f) directly contacting the collected organic salt with a heated gas stream while agitating the collected organic salt to reduce the moisture content of the dried organic salt to about 7% or less.

### BRIEF DESCRIPTION OF THE FIGURES

FIG. 1 is a graph showing the isomerization of cis-cyclohexane-1, 2-dicarboxylic acid calcium salt with increasing temperature.

### DETAILED DESCRIPTION OF THE INVENTION

In a first embodiment, the invention provides a process for making a high purity salt. The process generally comprises the steps of providing an organic compound (e.g., an organic acid or an anhydride thereof), providing a metal salt, adding the metal salt and organic compound to an aqueous medium, reacting the organic compound and metal salt to form an organic salt, collecting the organic salt from the aqueous medium, and directly contacting the collected organic salt with a heated gas stream while agitating the collected organic salt. The process is believed to yield an organic salt having an acceptably low moisture content and high purity (e.g., isomeric purity).

The process can utilize any suitable organic compound. Preferably, the organic compound is an organic acid, more preferably a carboxylic acid or an anhydride thereof. In one preferred embodiment, the organic compound is selected from the group consisting of cyclohexane-1,2-dicarboxylic acid anhydride, cyclohexane-1,2-dicarboxylic acid, and mixtures thereof. In a particularly preferred embodiment, the organic compound is selected from the group consisting of cis-cyclohexane-1,2-dicarboxylic acid anhydride, cis-cyclohexane-1,2-dicarboxylic acid, and mixtures thereof. In another preferred embodiment, the organic compound is selected from the group consisting of cis-cyclohexane-1,2-dicarboxylic acid anhydride. When the organic compound is selected from the group consisting of cis-cyclohexane-1,2-dicarboxylic acid anhydride, cis-cyclohexane-1,2-dicarboxylic acid, and mixtures thereof, the organic compound preferably contains relatively little of the corresponding trans isomers. Thus, in a preferred embodiment, the organic compound contains about 1.5 mol. % or less (e.g., about 1 mol. % or less) trans-cyclohexane-1,2-dicarboxylic acid anhydride or trans-cyclohexane-1,2-dicarboxylic acid.

The process can utilize any suitable metal salt. Preferably, the metal salt is selected from the group consisting of metal hydroxides, metal carbonates, metal nitrates, metal chlorides, metal carboxylates, and metal sulfates. Preferably, the metal salt comprises a metal selected from the group consisting of alkali metals and alkaline earth metals. In another preferred embodiment, the metal salt comprises a metal selected from the group consisting of alkaline earth metals. Suitable metal salts compounds include, but are not limited to, calcium hydroxide, sodium hydroxide, calcium carbonate, sodium carbonate, and mixtures thereof. In a preferred embodiment, the metal salt is selected from the group consisting of calcium hydroxide and calcium carbonate. In another preferred embodiment, the metal salt is calcium hydroxide. In yet another preferred embodiment, the metal salt is calcium carbonate. In another preferred embodiment, the metal salt is calcium nitrate. In another preferred embodiment, the metal salt is calcium sulfate. For metal carboxylates, preferred examples are, but not limited to, calcium acetate, calcium lactate, calcium formate, calcium gluconate, and calcium propionate.

In one step, the process entails the addition of the metal salt and the organic compound to an aqueous medium to produce a reaction mixture. The two compounds can be added to the aqueous medium or in any suitable order. In certain preferred embodiments (e.g., when a relatively insoluble metal salt is employed), the organic compound preferably is first added to the aqueous medium, and the resulting mixture is stirred or otherwise agitated until the organic compound has completely dissolved in the aqueous medium. In such an embodiment, the metal salt is then added, and the resulting mixture preferably is stirred or otherwise agitated to produce a substantially homogeneous reaction mixture. For example, when an insoluble metal salt is used, the mixture preferably is stirred or otherwise agitated to disperse the metal hydroxide compound in the reaction mixture.

The organic compound and the metal salt can be added to the aqueous medium in any suitable amounts. Preferably, to optimize purity of the resulting organic salt, the organic compound and the metal salt are added to the aqueous medium in stoichiometric amounts. However, when a soluble metal salt (e.g., sodium hydroxide) is employed, the metal salt can be added in a slight stoichiometric excess. In such cases, the excess soluble metal hydroxide compound will remain dissolved in the aqueous medium and can be easily separated from the target organic salt.

The reaction mixture preferably is heated to drive the reaction between the organic compound and the metal salt that forms the desired organic salt. The reaction mixture can be heated to any suitable temperature. In a preferred embodiment, the reaction mixture is heated to a temperature of about 30° C. or more, about 40° C. or more, about 50° C. or more, about 60° C. or more, about 80° C. or more, about 90° C. or more, about 100° C. or more, or the boiling point of the reaction mixture. In a preferred embodiment, the reaction mixture is heated to a temperature of about 65° C. to about 80° C. The reaction mixture can be heated to the desired temperature for any suitable amount of time. Preferably, the reaction mixture is heated to the desired temperature until the reaction between the organic compound and the metal salt is complete.

In some embodiments of the process, the product produced by the reaction between the metal salt and the organic compound is the desired organic salt. In such embodiments, the organic salt can be collected from the aqueous medium as described below. In other embodiments of the process, the product produced by the reaction between the metal salt and the organic compound can be further reacted or treated to produce the desired organic salt. For example, when the product produced by the reaction between the metal salt and the organic compound is a water-soluble intermediate organic salt, this water-soluble organic salt can be further reacted with a second salt in an ion exchange reaction to produce the desired organic salt. Suitable second salts for such ion exchange reactions include, but are not limited to, calcium salts, lithium salts, strontium salts, aluminum salts, and mixtures thereof.

Following the reaction steps, the target organic salt is collected from the aqueous medium. The target organic salt can be collected or separated from the aqueous medium using any suitable method. For example, the aqueous medium can be filtered to separate the target organic salt. Alternatively, the target organic salt can be removed from the aqueous medium using a centrifuge. The target organic salt can optionally be washed with water or inorganic solvent to remove unwanted byproducts.

Following separation and collection, the collected organic salt can be conveyed directly to the drying process, as described below. Alternatively, the collected organic salt can be granulated or milled to provide material having a more uniform particle size. While this step is not necessary for the described process, the efficiency of the drying step is improved when a more uniform particle size material is dried. Preferably, the collected organic salt is granulated to a particle size of about 1 mm or less, about 0.5 mm or less, about 0.4 mm or less, about 0.3 mm or less, or about 0.2 mm or less. Preferably, the collected organic salt is granulated to a particle size of between about 0.01 and 12 microns, preferably less than 10 microns.

Next, the collected organic salt is dried. The collected organic salt is directly contacted with a heated gas stream while at the same time being agitating to reduce the moisture content of the dried organic salt to about 7% or less.

Direct contact dryers provide the thermal energy required for moisture evaporation by direct contact of a heated gas stream with the product to be dried. Evaporation occurs in an adiabatic process with the particle temperature following the wet bulb temperature of the heated inlet gas stream, as long as the surface of the particle is wetted. It has been found that in indirect dryers (for example a simple oven), the collected organic salt acts as a very good insulator and the heat does not transfer well through the entire collection of salt and the middle section does not dry sufficiently. The insulatory properties of the calcium salt of cis-cyclohexane-1,2-dicarboxylic acid have been measured using the thermal conductivity method described in ISO 22007-2:2015—Plastics—Determination of Thermal Conductivity and Thermal Diffusivity—Part 2: Transient Plane Heat Source (hot disc) Method. The results are shown in Table 1:

TABLE 1
Thermal Conductivity of cis-Cyclohexane-1,2-dicarboxylic acid,
Calcium Salt

| | Thermal Conductivity |
|---|---|
| Sample | (W/m · K) |
| cis-Cyclohexane-1,2-dicarboxylic acid, | 0.0657 |
| Calcium salt | 0.0659 |
| | 0.0659 |
| | 0.0658 |
| | 0.0659 |
| Mean (W/m · K) | 0.0658 |
| Standard Deviation (W/m · K) | 0.0001 |
| RSD (%) | 0.0793 |

Based on these insulatory properties, the combination of both direct contact with the heated gas stream and agitation of the collection is very important to dry the collected organic salt to the desired dryness in a time and energy efficient manner.

The heated gas stream can have any suitable temperature, moisture content, gas content, and volumetric flow. The heated gas stream is introduced to the collected organic salt from a gas inlet. Preferably, the heated gas stream has a temperature of between about 120 and 250° C. at the gas inlet, more preferably between about 160 and 250° C. The heated gas stream may be ambient atmospheric composite or more preferably is at least 95% wt. nitrogen. Having a high concentration of nitrogen is preferred to reduce the chance of explosions due to static charge.

The collected organic salt can be exposed to the heated gas stream while at the same time being agitating for any suitable time. Preferably, the collected organic salt is exposed to the heat and agitation for a sufficient time to reduce the moisture content of the organic salt to about 7% or less, about 6% or less, about 5% or less, about 4% or less, about 3% or less, about 2% or less, or about 1.5% or less. The drying process may be a continuous or batch type process. In a continuous process the materials are continuously in motion, undergoing the direct heating by the heated gas stream and agitation. In a batch process, the drying is done to a set amount of collected organic salt at a time until dried to the desired amount, removed from the drying mechanism, and then a new batch of collect organic salt is added. At the preferred temperature described above, the collected organic salt is exposed to the heated gas stream for less than 20 minutes, more preferably less than 10 minutes, more preferably less than 5 minutes, more preferably less than 2 minutes, more preferably less than 1 minute. At the preferred temperature described above in a continuous type process, the collected organic salt is exposed to the heated gas stream for between about 0.5 seconds to 1 minute, more preferably less than about 20 seconds, more preferably less than about 10 seconds, more preferably less than about 2, more preferably less than about 1.8 seconds.

In one continuous process embodiment, the collected organic salt moves in a first direction within the continuous drying apparatus and the heated gas stream moves in the opposite direction. In another embodiment, both the collected organic salt and the heated gas stream more in the same direction through the (preferably continuous) drying apparatus. In other embodiment, the process involves gas being introduced to the collected organic salt in such a way to form a fluidized bed system.

In another embodiment, the drying apparatus contains rotating paddles, preferably mounted on a rod or screw. These paddles serve to move the collected organic salt through the apparatus and provide agitation to the collected organic salt. This apparatus with the paddles could be a batch or continuous process but is preferably a continuous process due to manufacturing efficiency. The rotating paddles comprise a paddle tip, defined as the part of the paddle furthest from the center axial that the paddles are attached to. In one embodiment, the paddle tip has a speed between about 10 and 20 meters/second.

In a preferred embodiment of the process, the organic salt produced by the claimed process is a cis-cyclohexane-1,2-dicarboxylate salt. In a particularly preferred embodiment, the organic salt is calcium cis-cyclohexane-1,2-dicarboxylate. As noted above, the process described herein is believed to be well-suited to the production of organic salts with relatively high isomeric purity (e.g., stereoisomeric purity). Thus, when the organic salt is a cis-cyclohexane-1,2-dicarboxylate salt, the organic salt preferably contains about 2.5 mol. % or less, about 1.5 mol. % or less, or about 1 mol. % or less, of trans-cyclohexane-1,2-dicarboxylate salts.

The powder can have any suitable particle size. However, in order to facilitate dispersion of the compounds in the molten polymer and prevent the formation of defects (e.g., white specks or bubbles) in the polymer composition, it is advantageous for the particles to have a relatively small particle size. In a preferred embodiment, the volume mean diameter (i.e., the D[4,3]) of the organic salt particles is about 40 μm or less, about 35 μm or less, about 30 μm or less, or about 25 μm or less. Further, the D₉₀ of the organic salt particles preferably is about 80 μm or less, about 75 μm or less, about 70 μm or less, about 65 μm or less, about 60 μm or less, or about 55 μm or less.

The particle size of the organic salt can be measured using any suitable technique. For example, the particle size of the powder can be measured via dynamic light scattering using one of the many commercially available instruments designed for such measurements. When a dynamic light scattering technique is used, a representative sample of the particles generally is dispersed in a liquid medium and a sample of this liquid medium is introduced into the dynamic light scattering instrument. Any suitable liquid medium can be used, but water generally is the preferred medium. In order to facilitate dispersion of the particles in the liquid medium, a surfactant, preferably a non-ionic surfactant (e.g., an octylphenol surfactant), can be added to the water and the resulting mixture (i.e., water, surfactant, and particles) can be stirred for a sufficient time for the particles to disperse (e.g., for 1-5 minutes).

The particle size of the organic salt particles can be the same (e.g., in terms of volume mean diameter, D₉₀, or both) as the particle size of the organic salt particles described above. Alternatively, the particle size of the organic salt particles can be smaller (e.g., in terms of volume mean diameter, D₉₀, or both) than the particle size of the organic salt particles. In a preferred embodiment, the volume mean diameter (i.e., the D[4,3]) of the organic salt particles is about 40 μm or less, about 35 μm or less, about 30 μm or less, about 25 μm or less, about 20 μm or less, about 15 μm or less, about 10 μm or less, or about 7.5 μm or less. Further, the D₉₀ of the organic salt particles preferably is about 80 μm or less, about 75 μm or less, about 70 μm or less, about 65 μm or less, about 60 μm or less, about 55 μm or less, about 50 μm or less, about 45 μm or less, about 40 μm or less, about 35 μm or less, about 30 μm or less, about 25 μm or less, about 20 μm or less, about 15 μm or less, about 10 μm or less, or about 7.5 μm or less.

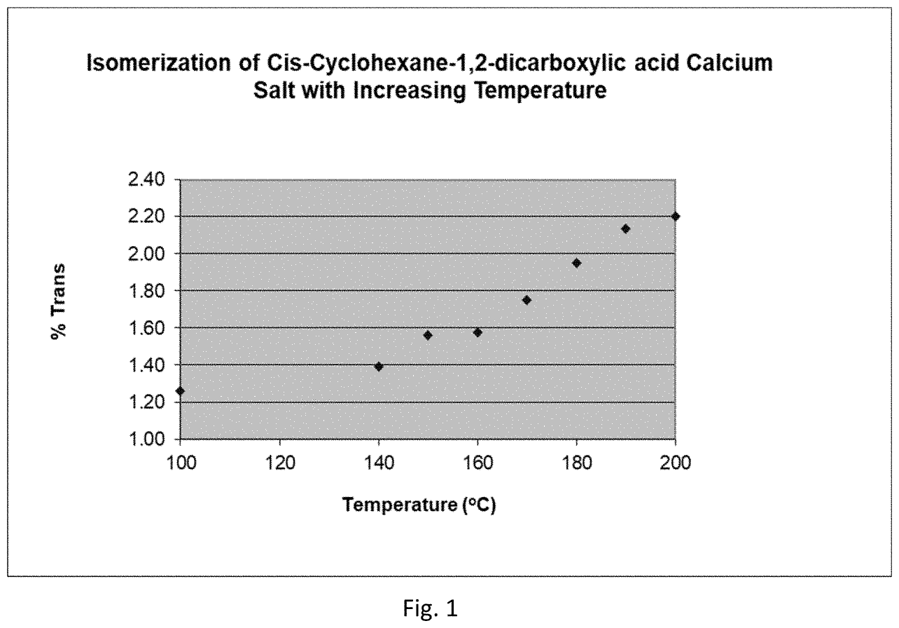

Exposure of the product to extremely high heat for extended periods of time results in isomerization. FIG. 1 is a graph showing the isomerization of cis-cyclohexane-1, 2-dicarboxylic acid calcium salt with increasing temperature.

For this graph, the isomer content was measured after every hour at the selected temperature, with the sample being dried in an open pan directly in contact with the heat source.

Direct gas drying processes do not result in significant isomerization because of the limited residence time and excellent thermal transfer to the individual particles in the gas stream. Isomer contents of less than 1% trans are routinely achieved using these specific drying techniques described above.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the subject matter of this application (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the subject matter of the application and does not pose a limitation on the scope of the subject matter unless otherwise claimed. No language in the specification should be construed as indicating any non-claimed element as essential to the practice of the subject matter described herein.

Preferred embodiments of the subject matter of this application are described herein, including the best mode known to the inventors for carrying out the claimed subject matter. Variations of those preferred embodiments may become apparent to those of ordinary skill in the art upon reading the foregoing description. The inventors expect skilled artisans to employ such variations as appropriate, and the inventors intend for the subject matter described herein to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

What is claimed is:

1. A process for making a high purity salt, the process comprising the steps of:
(a) providing an organic compound selected from the group consisting of cis-cyclohexane-1,2-dicarboxylic acid anhydride, cis-cyclohexane-1,2-dicarboxylic acid, and mixtures thereof;
(b) providing a metal salt selected from the group consisting of metal hydroxides, metal carbonates, metal nitrates, metal chlorides, metal carboxylates, the metal salt comprising a metal selected from the group consisting of alkali metals and alkaline earth metals;
(c) adding the metal salt and the organic compound to an aqueous medium to produce a reaction mixture;
(d) heating the reaction mixture to a temperature of about 65° C. to about 80° C. to react the organic compound and the metal salt and form an organic salt;
(e) collecting the organic salt from the aqueous medium;
(f) directly contacting the collected organic salt with a heated gas stream while agitating the collected organic salt to reduce the moisture content of the dried organic salt to about 7% or less.

2. The process of claim 1, wherein the organic compound is cis-cyclohexane-1,2-dicarboxylic acid anhydride.

3. The process of claim 1, wherein the organic compound contains about 1.5 mol. % or less trans-cyclohexane-1,2-dicarboxylic acid anhydride or trans-cyclohexane-1,2-dicarboxylic acid.

4. The process of claim 1, wherein the heated gas stream is introduced to the collected organic salt from a gas inlet, wherein the heated gas stream at the gas inlet has a temperature of between about 160 and 250° C.

5. The process of claim 1, wherein the heated gas stream at inlet comprises at least about 95% wt. nitrogen.

6. The process of claim 1, wherein the collected organic salt is exposed to the direct contact of a heated gas stream and agitation of step f. for between about 0.1 to 20 seconds.

7. The process of claim 1, wherein the agitating the collected organic salt comprises the use of rotating paddles, wherein the rotating paddles comprise a paddle tip, wherein the paddle tip has a speed between about 10 and 20 meters/second.

8. The process of claim 1, wherein the collected organic salt moves in a first direction and the heated gas stream moves in the opposite direction.

9. The process of claim 1, wherein step (f) is a continuous process having a residence time of between about 0.5 seconds and 1 minute.

10. The process of claim 1, wherein step (f) is a batch process.

11. The process of claim 10, wherein the dried organic salt contains about 1 mol. % or less of trans-cyclohexane-1,2-dicarboxylate salts.

12. A process for making a high purity salt, the process comprising the steps of:
(a) providing an organic compound selected from the group consisting of cis-cyclohexane-1,2-dicarboxylic acid anhydride, cis-cyclohexane-1,2-dicarboxylic acid, and mixtures thereof;
(b) providing a calcium salt;
(c) adding the calcium salt and the organic compound to an aqueous medium to produce a reaction mixture;
(d) heating the reaction mixture to a temperature of about 65° C. to about 80° C. to react the organic compound and the metal salt and form an organic salt;
(e) collecting the organic salt from the aqueous medium;
(f) directly contacting the collected organic salt with a heated gas stream while agitating the collected organic salt to reduce the moisture content of the dried organic salt to about 7% or less.

13. The process of claim 12, wherein the organic compound is cis-cyclohexane-1,2-dicarboxylic acid anhydride.

14. The process of claim 12, wherein the organic compound contains about 1.5 mol. % or less trans-cyclohexane-1,2-dicarboxylic acid anhydride or trans-cyclohexane-1,2-dicarboxylic acid.

15. The process of claim 12, wherein the organic salt is calcium cis-cyclohexane-1,2-dicarboxylate.

16. The process of claim 15, wherein the organic salt contains about 1 mol. % or less of trans-cyclohexane-1,2-dicarboxylate salts.

17. The process of claim 12, wherein the heated gas stream is introduced to the collected organic salt from a gas inlet, wherein the heated gas stream at the gas inlet has a temperature of between about 160 and 250° C.

18. The process of claim 12, wherein the heated gas stream at inlet comprises at least about 95% wt. nitrogen.

19. The process of claim 12, wherein the collected organic salt is exposed to the direct contact of a heated gas stream and agitation of step f. for between about 0.1 to 20 seconds.

20. The process of claim 12, wherein the agitating the collected organic salt comprises the use of rotating paddles, wherein the rotating paddles comprise a paddle tip, wherein the paddle tip has a speed between about 10 and 20 meters/second.

21. The process of claim 12, wherein the collected organic salt moves in a first direction and the heated gas stream moves in the opposite direction.

22. The process of claim 12, wherein step (f) is a continuous process having a residence time of between about 0.5 seconds and 1 minute.

23. The process of claim 12, wherein the dried organic salt contains about 1 mol. % or less of trans-cyclohexane-1,2-dicarboxylate salts.
